(19) United States
(12) Patent Application Publication
(10) Pub. No.: US 2010/0075319 A1
(43) Pub. Date: Mar. 25, 2010

(54) Blocking Agents Comprising Non-Natural Nucleic Acids and Detection Methods Using such Blocking Agents

(76) Inventor: Jesper Lohse, Copenhagen (DK)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/516,685

(22) PCT Filed: Nov. 29, 2007
(86) PCT No.: PCT/IB07/04387

§ 371 Date: May 28, 2009

Related U.S. Application Data
(60) Provisional application No. 60/861,955, filed on Dec. 1, 2006.

(51) Int. Cl. C12Q 1/68

(52) U.S. Cl. 435/6

(57) ABSTRACT

This invention relates to nucleic acid analog blocking agents that may reduce nonspecific interactions between components of a biological or chemical detection assay. The blocking agents may help to reduce the background observed in biological or chemical detection assays, such as in situ assays and blots, and may thus enhance the signal to noise in the assays. The invention also encompasses sets of nucleic acid analog segments, for instance, made from PNA and/or non-natural bases, which may act as blocking agents and/or detection reagents, reagent kits containing those sets, and related methods of detection. In some embodiments, the blocking agents are designed such that they block one or more sets of complementary strands of nucleic acids on a detection reagent or in a sample, but do not hybridize to each other. In some embodiments, the blocking agents may block genomic repeat sequences such as one or more of Alu repeats, Kpn repeats, di-nucleotide repeats, tri-nucleotide repeats, penta-nucleotide repeats, and hexa-nucleotide repeats.

### RELATED APPLICATIONS

This application claims priority to U.S. Provisional Patent Application No. 60/861,955, filed Dec. 1, 2006, which is incorporated herein by reference.

### FIELD OF THE INVENTION

This invention relates to nucleic acid blocking agents that may reduce interactions between components of a biological or chemical detection assay.

### BACKGROUND AND SUMMARY OF THE INVENTION

The blocking agents of this invention may help to reduce the background observed in biological or chemical detection assays, such as in situ assays and blots, and may thus enhance the signal to noise in the assays. The invention also encompasses sets of nucleic acid analog segments, for instance, made from PNA and/or non-natural bases, which may act as blocking agents and/or detection reagents, reagent kits containing those sets, and related methods of detection.

Many biological detection assays are performed with samples that naturally contain DNA and/or RNA molecules, for instance cell samples, tissue samples, blots, and the like. Examples of detection assays performed with samples that may contain nucleic acids include in situ hybridization (ISH), immunohistochemistry (IHC), immunocytochemistry (ICC), flow cytometry, enzyme immuno-assays (EIA), enzyme linked immuno-assays (ELISA), Western, Southern, and Northern blots, as well as methods of labeling inside electrophoresis systems or on surfaces or arrays, and precipitation methods, among others. Such detection formats may be useful in research as well as in diagnosing diseases or conditions. Certain detection assay systems may also be designed such that the detection reagents interact together via nucleic acid hybridization. (See U.S. Provisional Application No. 60/695,410 and PCT Application No. PCT/IB2006/003130 for examples.)

Blocking agents may be included in a biological or chemical detection assay, either applied in a separate step, or together with one or more detection reagents. Such agents can, in some cases, increase the signal to noise and reduce false positive signals. Some blocking agents, such as Cₒt-DNA, salmon sperm DNA, total human DNA, and ALU-PNAs, may block binding between detection reagents and nucleic acids in a sample. Similarly, blocking agents such as bovine serum albumin, ovalbumin, or milk proteins may be used to reduce unwanted nonspecific protein-ligand interactions in an assay. The above blocking agents are generally intended to reduce non-specific interactions between assay components and the sample and to block genomic repeat sequences such as Alu repeats that may be present on genomic DNA in a sample, or in other detection reagents such as nucleic acid probes.

Nucleic acids in a sample may include double-stranded and complementary sequences. Conventional blocking agents may block only one strand of a double-stranded non-target DNA. Yet blocking both complementary strands with natural nucleic acid blocking agents is complicated by the fact that those blocking agents themselves would also need to be complementary to each other. Hence, the complementary blocking agents would tend to hybridize together and may be relatively unreactive and inefficient.

One way to attempt to solve that problem is to heat and cool the sample to denature and renature nucleic acid duplexes. But heating a sample, even to relatively modest temperatures, can interfere with certain detection systems by denaturing other detection reagents such as proteins or detectable labels. For example, stringent, high temperature washing procedures may adversely affect some probe-target interactions, such as antibody-antigen interactions, either by causing the antibody or antigen to unfold or by reducing the binding affinity for the antibody and antigen. Stringent, high temperature washes may also adversely affect labels such as R-Phycoerythrin (RPE) or alkaline phosphatase (AP). Hence, the instant inventors have found that new types of blocking agents may help to improve biological detection schemes, for instance by reducing or eliminating the need for those stringent, high temperature washing conditions.

The present invention includes sets of nucleic acid segments that specifically hybridize to complementary nucleic acid segments but do not specifically hybridize to each other. Those segments use non-natural bases, for example, in patterns such that two blocking agents may hybridize specifically to complementary natural nucleic acid sequences without hybridizing significantly to each other.

In some embodiments, the blocking agents are designed such that they block one or more sets of complementary strands of nucleic acids on a detection reagent or in a sample, but do not hybridize to each other. The blocking agents may be incorporated into a set of detection reagents that includes, for example, two other nucleic acid segments that each bind to complementary strands of target nucleic acids in the sample. In some embodiments, the blocking agents may block genomic repeat sequences such as one or more of Alu repeats, Kpn repeats, di-nucleotide repeats, tri-nucleotide repeats, penta-nucleotide repeats, and hexa-nucleotide repeats, including both strands of those repeats. In some embodiments, repeat sequences are also present on detection reagents in addition to unique sequences that bind to the intended targets in the sample. In such cases, the blocking agents may also block the repeat sequences present on the detection reagents as well as those present in the sample.

Some detection schemes may also use nucleic acid hybridization to physically link two or more detection reagents together. For example, an antibody bound to a target in a sample may be linked to a detectable label, such as a fluorophore, by nucleic acid hybridization if the antibody and fluorophore are each attached to complementary nucleic acid segments. (See U.S. Provisional Application No. 60/695,410 and PCT Application No. PCT/IB2006/003130 for examples.) Those complementary nucleic acid sequences in the detection reagents can be short segments of, for example, 5, 6, 8, 10, 12, 14, or 16 nucleobases, and may themselves be complementary or partially complementary to other nucleic acids present in the sample. Hence, blocking those non-target sequences in the sample may help improve the performance of such detection assays, particularly if the blocking agents are complementary to the non-target sequences in the sample but are not complementary to (i.e. do not significantly bind to) the sequences of the detection reagents.

The sets of nucleic acid blocking agents described herein, comprising at least one non-natural nucleobase, may block two complementary, non-target nucleic acid sequences in a sample, which otherwise might bind to the complementary nucleic acid segments in the detection reagents and interfere with the detection protocol. In some embodiments, the blocking agents may also bind to complementary sequences on detection reagents that might interfere with the performance or efficiency of those detection reagents, e.g. genomic repeat sequences. Yet the instant blocking agents may also not significantly bind to each other or to the nucleic acid sequences of the detection reagents that are intended to bind to specific targets.

Some detection experiments involve multiple sets of interacting nucleic acid segments. For example, some experiments also include an amplification layer to enhance the signal resulting from detection of a target. For instance, a target may be detected by a primary antibody specific for that target. Then a secondary antibody may be added, which specifically binds to the primary antibody. As a result, many secondary antibodies are bound to each primary antibody. If a detectable label is attached to the secondary antibody, each target molecule becomes associated with multiple labels rather than only one or a few labels, thus strengthening its overall signal. Similarly, amplification may also occur via adaptor molecular entities that use nucleic acid base-pairing to recognize a probe. In such systems, the recognition of primary and secondary detection reagents occurs via specific nucleic acid hybridization. Some detection assays may use two or more layers of amplification, or may use more than one type of amplification layer. The instant blocking agents may also be useful in reducing or eliminating unwanted interactions between an amplification layer and the sample so that the amplification layer binds more specifically to its intended probe and target.

### DETAILED DESCRIPTION OF THE INVENTION

### Definitions

Sample, as used herein, refers to any composition potentially containing a target that may be detected. Target, as used herein, refers to any substance present in a sample that is intended to be detected in a detection assay, while a non-target is a substance that is not intended for detection. Detection assay, as used herein, refers to any method of detecting a target in a sample. A detection reagent, as used herein refers to a component of a detection assay which is involved in binding to a target and detecting the binding to the target. Examples include probes, detectable labels, and adaption units, and molecular entities comprising them, as described below.

According to this invention, a probe comprises any substance that is capable of recognizing a target in the sample. In some embodiments of this invention, the probe is part of a larger molecular entity, referred to as a recognition unit. In some embodiments, the recognition unit also comprises at least one nucleic acid analog segment that recognizes other detection reagents in the assay.

The terms recognize, recognition, or recognizing, etc., as used herein, mean an event in which one substance, such as a probe or recognition unit comprising a probe, directly or indirectly binds to a target in any way such that the interaction with the target may be detected.

As used herein, a detectable label is a substance which allows for detection of the bound target in the sample, such as through color, fluorescence, radioactivity, or some other measurement means. In some embodiments, the detectable label is part of a larger molecular entity referred to as a detection unit. In some embodiments of this invention, a detection unit comprises at least one nucleic acid analog segment used to link the detection unit to other detection reagents such as a recognition unit or probe, or an adaptor unit.

An adaptor unit, as used herein, means a substance that is capable of linking a recognition unit to a detection unit. In some embodiments of this invention, an adaptor unit comprises at least two different nucleic acid analog segments, one of which specifically hybridizes to a recognition unit, and the other of which specifically hybridizes to a detection unit, serving to link them together.

An amplification layer or reagent for amplification, as used herein refers to a molecule or molecular entity which binds to a probe or recognition unit or to an adaptor unit in such a way as to amplify the resulting signal from the binding of probe to target. For example, multiple amplification reagents may bind to the probe, such that each probe becomes associated with multiple detectable labels. An amplification layer or reagent may comprise a detectable label or may recognize another detection reagent carrying the detectable label.

The terms specifically hybridizes, specific hybridization, and the like, as used in this application, mean the formation of hydrogen bonds between two or more nucleic acid segments or nucleic acid analog segments under at least low stringency conditions. Non-limiting examples of the formation of hydrogen bonds between the segments include the formation of Watson-Crick, wobble, and Hoogsteen base-pair geometries, such as to form double strands.

Antibody, as used herein, means an immunoglobulin or a fragment thereof, and encompasses any polypeptide comprising an antigen-binding site regardless of the source, method of production, and other characteristics.

An antigen, as used herein, refers to any substance recognized by an antibody.

As used herein, the terms base and nucleobase refer to any purine-like or pyrimidine-like molecule that may be comprised in a nucleic acid segment or nucleic acid analog segment.

A non-natural base, as used herein, means any nucleobase other than the natural bases: Adenine, A; Guanine, G; Urasil, U; Thymine, T; Cytosine, C.

A non-natural backbone unit includes any type of backbone unit to which a nucleobase may be attached that is not a ribose-phosphate (RNA) or a deoxyribose-phosphate (DNA) backbone unit.

As used herein, a nucleic acid analog segment means any oligomer, polymer, or polymer segment, comprising at least one monomer that comprises at least one non-natural base and/or at least one non-natural backbone unit. A natural nucleic acid segment means any oligomer, polymer, or polymer segment consisting of one or more of the natural bases A, T, U, G, and C, such that it's base sequence is entirely made up of natural bases. A natural nucleic acid segment may have at least one non-natural backbone unit, however. A nucleic acid segment, more generally, encompasses both a natural nucleic acid segment and a nucleic acid analog segment.

As used herein, all numbers are approximate, and may be varied to account for errors in measurement and rounding of significant digits.

### BRIEF DESCRIPTION OF THE DRAWINGS

FIG. 1: Example of an in situ or blotting assay in which a non-target DNA or RNA sequence in the sample is blocked by a blocking agent, allowing the probe to hybridize specifically to target sequence. Panel B shows a similar assay in which blocking agents block two complementary non-target strands, allowing probes to hybridize specifically to target strands.

FIG. 2: Example of a detection assay that utilizes specific hybridization between detection reagents to link the target to a detectable label. (See U.S. Provisional Application No. 60/695,410 and PCT Application No. PCT/IB2006/003130 for examples.) In such an assay, a blocking agent may be helpful in blocking DNA or RNA sequences in the sample that are complementary or partially complementary to those detection reagents, so that the detection reagents may hybridize specifically to each other.

FIG. 3: Example of a detection assay utilizing specific hybridization to link target to detectable label, in which the assay also contains an amplification layer for signal enhancement. (See U.S. Provisional Application No. 60/695,410 and PCT Application No. PCT/IB2006/003130 for examples.)

FIG. 4: Double-stranded DNA (dsDNA) probes contain unique sequences (solid lines) as well as repetitive sequences (e.g. Alu sequences, dotted lines). By adding Cₒt DNA, the repetitive Alu sequences are blocked, leaving the unique sequences to hybridize to the complementary dsDNA target. Alu PNAs target alternating Alu sequences on the two strands. Blocking agents with non-natural bases would allow targeting of both strands of the repetitive sequence.

FIG. 5: This figure depicts a detection assay in which the target is linked to a detectable label via nucleic acid hybridization between two sequences on the detection reagents, denoted L1 and L2. To reduce the chance that L1 and L2 could form unwanted interactions with non-target complementary or partially complementary natural DNA or RNA sequences D1 and D2 in the sample, B1 and B2 blocking agents are designed to form stronger interactions with D1 and D2 than the interactions formed between D1 and D2 and L1 and L2. Yet B1 and B2 do not hybridize significantly to each other. (See the arrows on the left panel, whose width depicts relative binding affinities.) In some embodiments, blocking agents such as B1 and B2 cause sequences D1 and D2 in the sample to become blocked, allowing L1 and L2 to interact more efficiently together. In this schematic, interaction between L1 and L2 causes an antigen to become associated with a fluorescent or colored detection label.

FIG. 6: Example sequences of a group of blocking agents B1 and B2 and hybridizing detection reagents L1 and L2, such as could be used in the assay depicted in FIG. 5, for instance, to block unwanted interactions between detection reagents L1 and L2 with non-target natural DNA sequences D1 and D2 in a sample. In the schematic, the blocking agents B1 and B2 bind to complementary DNAs D1 and D2 in the sample, but do not bind to each other (arrows on right panel show repulsion between bases). The detection reagents L1 and L2 specifically hybridize in the assay in order to link a target to a detectable label. However, L2 has a weak affinity for sample DNA sequence D1 while L1 has weak affinity for D2. B1 and B2 may be designed to compete with L1 and L2 for binding to D1 and D2, thus freeing L1 and L2 to hybridize to each other more efficiently in the assay. For instance, B1 and B2 might bind to D1 and D2 with faster kinetics than L1 and L2 bind to D1 and D2. Or, B1 and B2 might bind with higher affinity to D1 and D2 than L1 and L2 bind to D1 and D2.

FIG. 7: Depiction of base-pairing between non-natural and natural bases which may be utilized to design blocking agents.

FIGS. 8-10: Examples of natural and non-natural bases and base pairs that may be used in conjunction with this invention.

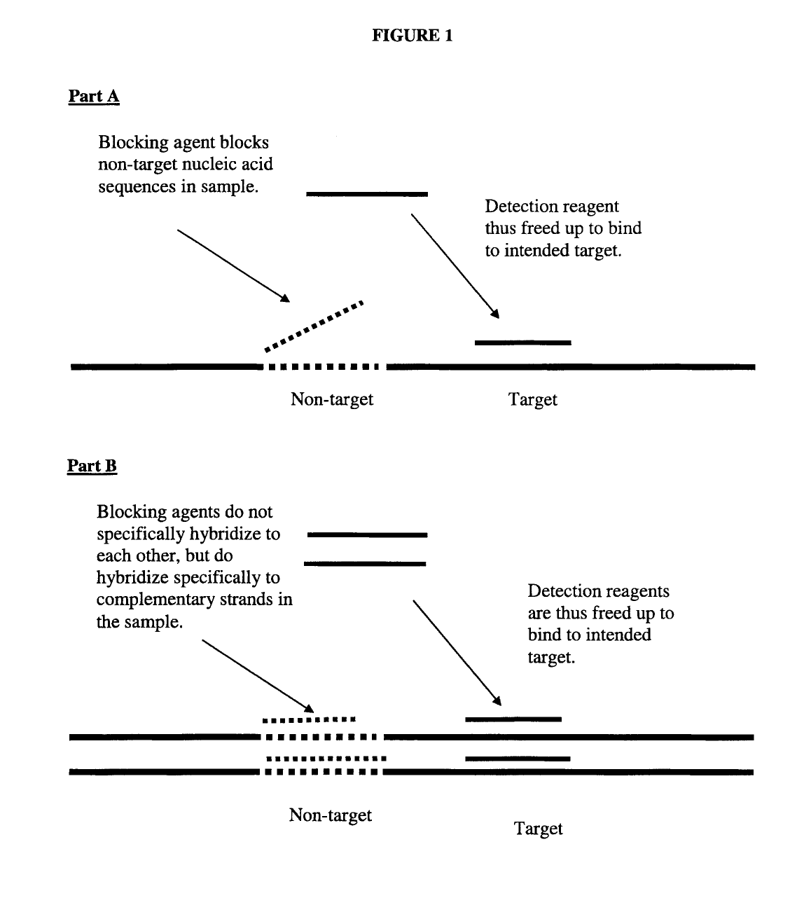

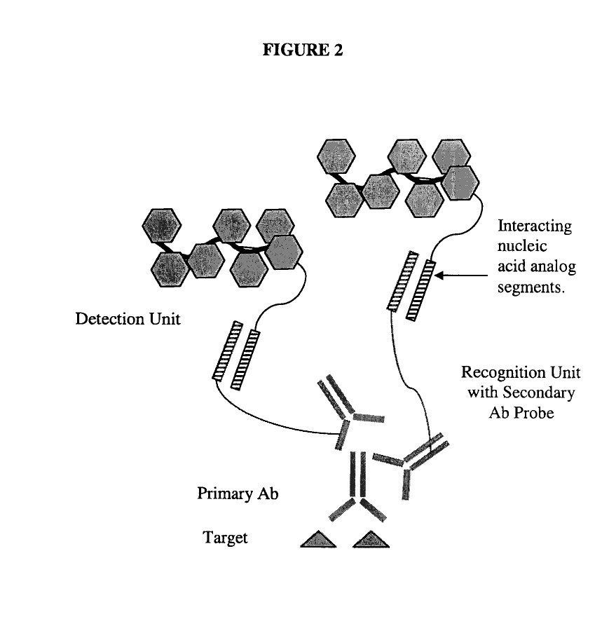

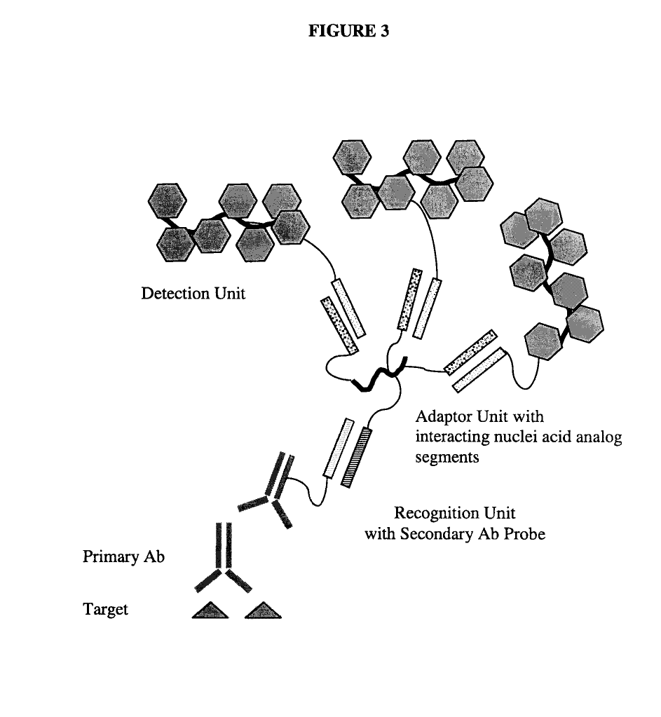

### DESIGN OF BLOCKING AGENTS AND CORRESPONDING DETECTION REAGENTS

The present invention takes advantage of the wider range of base-pairing schemes available through the use of non-natural bases in both detection reagents and blocking agents. For example, some non-natural bases can be used to make detection reagents with reduced affinity for non-target DNA or RNA sequences in a sample as compared to conventional detection reagents made only from the natural bases A, C, G, T, and U. In some embodiments, two complementary, non-natural bases may hybridize to each other more strongly than either base hybridizes to any of the natural A, C, G, T, or U bases.

### Base-Pairing Schemes

In some embodiments of the invention, the base-pairing schemes for the blocking agents may be chosen based on the intended pairing between a target sequence and a detection reagent in an assay, or based on an intended hybridization between two detection reagents. For example, if one desires to detect a target DNA sequence, D1, in a sample with a probe L1, one may design a blocker B1 that binds to the D1 sequence itself, portions of that sequence, such as repetitive sequence elements, or to sequences that are partially complementary to D1. Such blocking may allow L1 to hybridize to D1 more specifically in some embodiments.

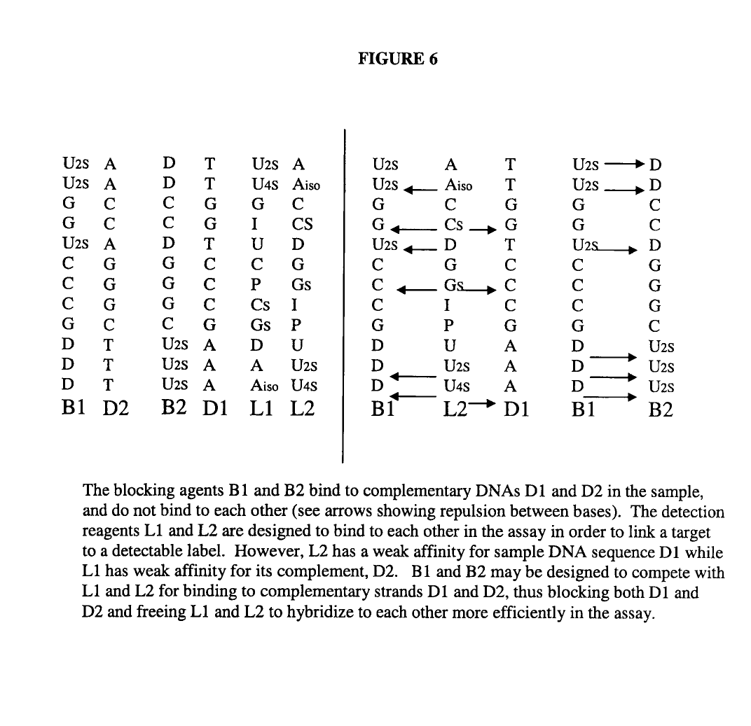

If one intends that two detection reagents L1 and L2 interact by forming duplexes with each other, then one might wish to construct two blocking agents, B1 and B2, that interact with complementary nucleic acid sequences in a sample to which L1 and L2 might unintentionally bind, D1 and D2. (See FIG. 6 for an example.) At the same time, one may which to constrain the sequences of B1 and B2 such that they (1) do not significantly hybridize to each other; and/or (2) do not significantly hybridize to the detection reagents L1 or L2, but such that they do bind efficiently to D1 and D2.

While the blocking agents can be designed so that they do not specifically hybridize to the detection reagents in the assay, more freedom of design of the detection reagents is obtained if this restraint is relieved. In such embodiments, potentially stronger binding and more varied pairs of non-natural nucleic acid detection reagents can be prepared. Thus, in some embodiments where binding can occur between the blocking agents and the detection reagents, the blocking agents are applied in a separate pre-blocking step and thus are not mixed together with the detection reagents. In multi layer and/or multiplexing protocols this may simplify the experimental design, as one could, for instance, begin the assay by blocking all potential nucleic acid binding sites in the sample with a cocktail of non-natural nucleic acid binding agents prior to proceeding with the assay and adding the detection reagents.

### Non-Natural Base-Pairing Interactions

A variety of bases are compatible with the instant invention. This section provides other examples and also further illustrates the interactions between the previously described non-natural and natural bases.

For example, substituting one amino group with a Hydrogen or small Halogen (collectively “h”) on one of the bases creates six types of bases:

2-amino-6-“h”-purines

6-amino-2-“h”-purines

6-oxo-2-“h”-purines

2-oxo-4-“h”-pyrimidines

2-oxo-6-“h”-purines

4-oxo-2-“h”-pyrimidines

These will form two hydrogen bond base pairs with non-thiolated and thiolated bases; respectively:

2,4 dioxo and 4-oxo-2-thioxo pyrimidines

2,4 dioxo and 2-oxo-4-thioxo pyrimidines

4-amino-2-oxo and 4-amino-2-thioxo pyrimidines

6-oxo-2-amino and 6-thioxo-2-amino purines

2-amino-4-oxo and 2-amino-4-thioxo pyrimidines

6-oxo-2-amino and 6-thioxo-2-amino purines.

### Whereas:

4-oxo-2-thioxo pyrimidines will not pair with 2,6-diaminopurines or 2-amino-6-“h”-purines;

2-oxo-4-thioxo pyrimidines will not pair with 2,6-diaminopurines or 6-amino-2-“h”-purines;

4-amino-2-thioxo pyrimidines will not pair with 2-amino-6-thioxopurines or 2-“h”-6-thioxopurines;

6-thioxo-2-amino purines will not pair with 4-amino-2-oxopyrimidines or 4-amino-2-thioxopyrimidines;

2-amino-4-thioxo pyrimidines will not pair with 2-thioxo-6-aminopurines or 2-thioxo-6-“h”-purines; and

6-thioxo-2-amino purines will not pair with 2-thioxo-6-aminopurines or 2-thioxo-6-“h”-purines.

In each of the above cases, two bases A and B have three mutual hydrogen bonds and form a very stable mutual pair. By substituting one of the hydrogen bonding amino groups on one of the bases, say A, with hydrogen or a small halogen, we get a new base C, and by substituting the facing carbonyl on B, we get a thiocarbonylated base D.

C will now pair with both B and D, forming two stable, two hydrogen bond pairs. A will however not pair with D (or other analogues of B with a thiocarbonyl facing an amino group in A). For example, 2-ThioUracil does not pair with Diaminopurine but pairs with Adenine, while Adenine pairs with both Uracil and 2-ThioUracil, i.e. these 4 bases follow the described pattern, A=Diaminopurine, B=Uracil, C=Adenine, D=2-ThioUracil.

In addition, 2-Oxo-pyrimidine pairs with both Guanine and ThioGuanine (Gs). Thiocarbonyls, such as Cs, due to their increased size, repel bases with facing amino groups, despite two other mutual hydrogen bonds (disallowing, for example, D:U2s and D:U4s) but may pair via two hydrogen bonds with bases that have a hydrogen facing the thiocarbonyls, allowing additional base pairs that also contribute to complex stability. Accordingly, Cs pairs stably with I but not with G. Some base pairs, such as I:P, only share a single central hydrogen bond, such as I:P, and is expected to be significantly weaker.

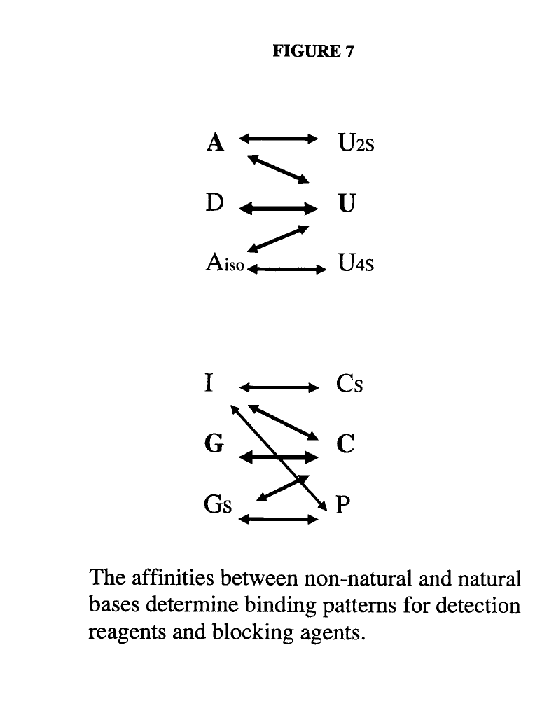

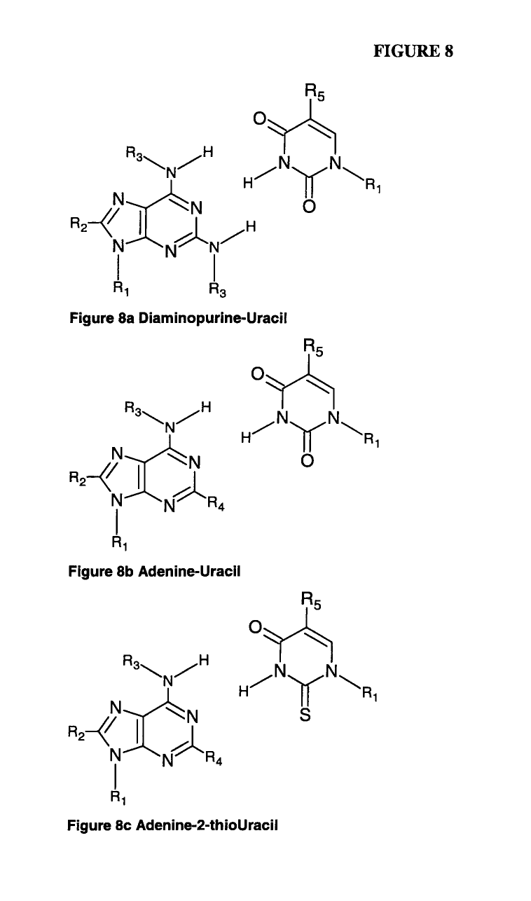

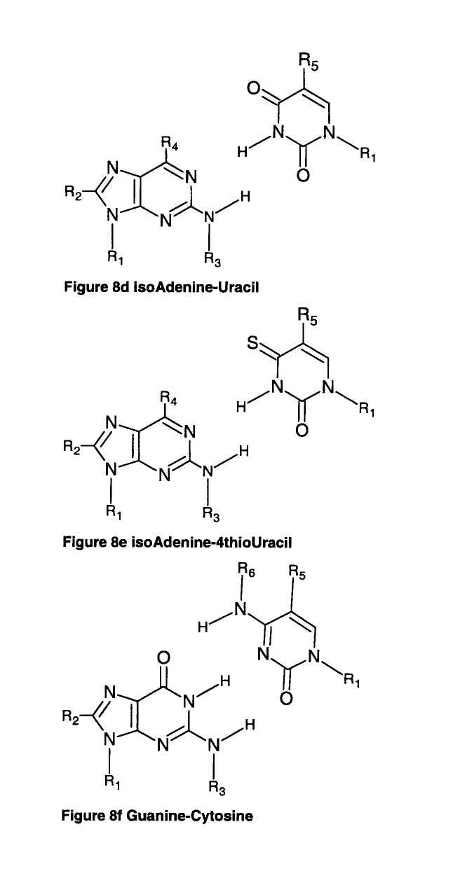

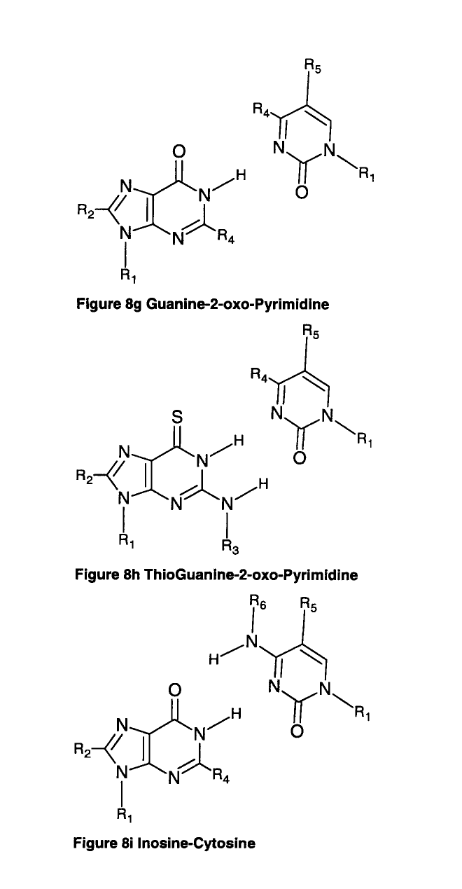

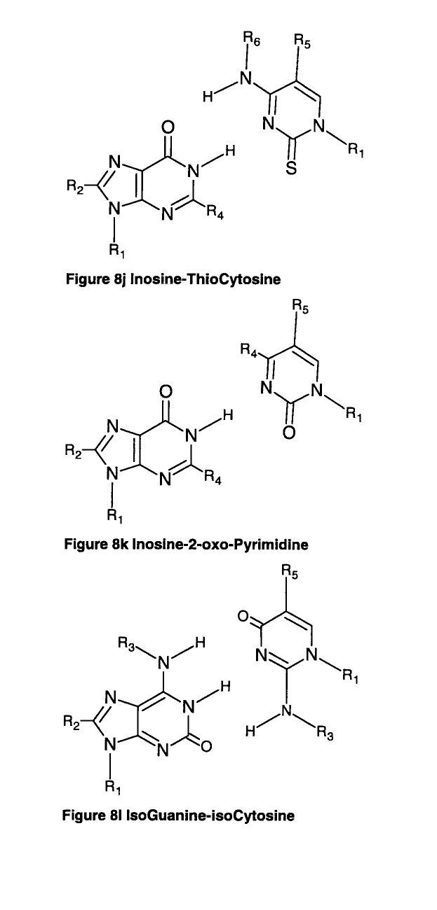

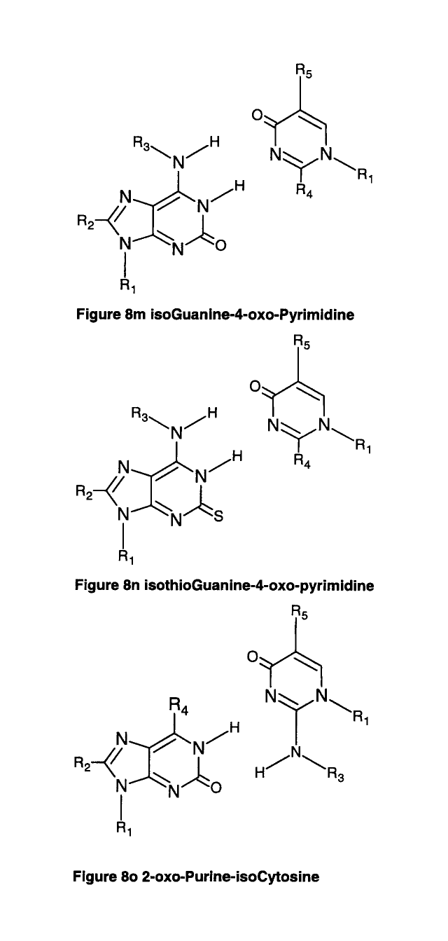

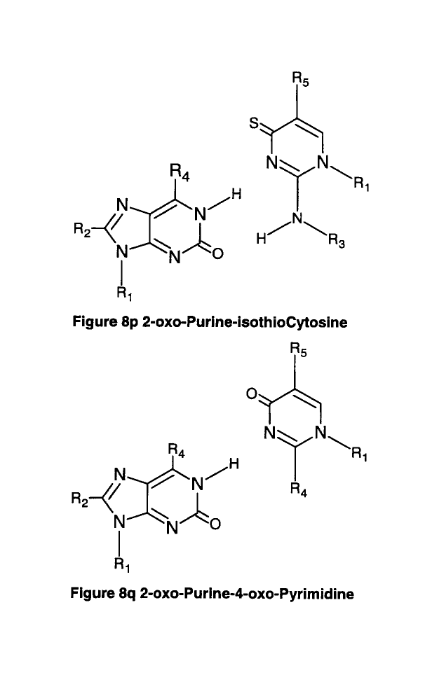

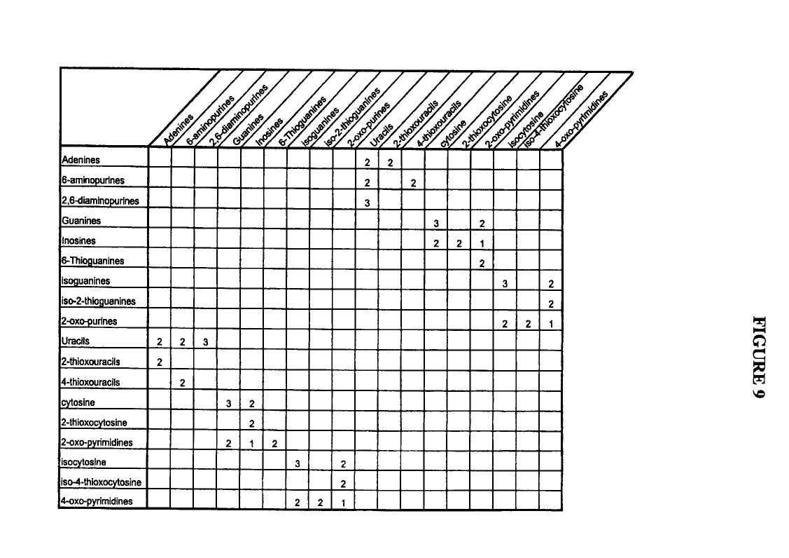

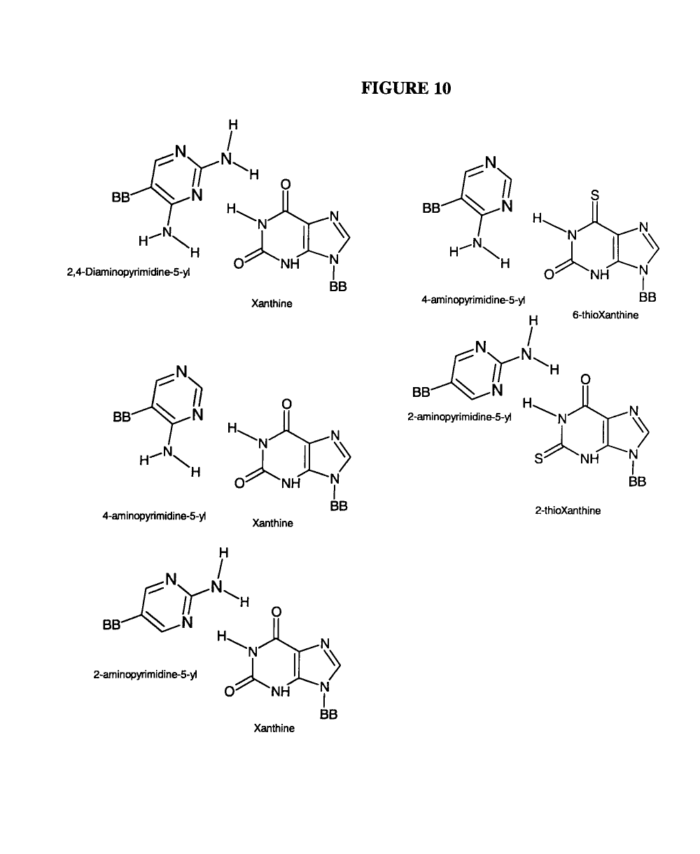

In some embodiments, detection reagents comprising L1 and L2 may be intended to interact during the detection process, and may be designed to have moderate affinity for each other, for example, by incorporating non-natural bases into each sequence. For instance, in addition to a simple A-U or A-T base-pair, as found in natural nucleic acids, the L1 and L2 segments could interact via one or more non-natural base-pairs, such as A-U2s, D-U, isoA-U, and isoA-U4s, for instance, where D=diaminopurine; U2s=2-thio-uracil; U4s=4-thio-uracil, and isoA=iso-adenosine, (See FIGS. 6-8.) If L1 contains an A, D, or isoA residue at a given position in its sequence, L1 might inadvertently interact with DNA or RNA sequences that contain a corresponding natural T or U residue. The complementary L2 containing a U, U2s, or U4s, might interact with an A residue on a nucleic acid of the sample. To block such interactions, blockers B1 and B2 may be designed to interact with those natural A and U/T base-pairs in the sample, but to avoid base-pairing interactions with each other. For example, the pairings D-U2s, isoA-U2s, and D-U4s do not produce stable base-pair hydrogen bonding schemes and may, in fact, repel each other, thus diminishing the affinity between B1 and B2 but allowing each of B1 and B2 to bind to D1 and D2. (See FIGS. 6-8.) Segments B1 and B2 may also be designed such that they have limited or no significant affinity for L1 and L2, even though L1 and 12 also have some affinity for D1 and D2, again taking advantage of the wider scope of base pairing interactions allowed by using non-natural bases.

Similarly, L1 and L2 may be designed to interact through a G-C-like pairing at a particular location, such as G-C, I-Cs, or Gs-P, where I=inosine; Cs=thio-cytosine; Gs=thio-guanine; and P=pyrimidine. Blockers B1 and B2 may be designed to prevent the G-C, I-Cs, or Gs-P pairings in L1 and L2 from interacting with G-C base pairs in sample DNA or RNA, but nonetheless, to avoid significantly binding to each other or to L1 and L2. Such blocking agents may include a Cs and a G residue at the pairing location in B1 and B2, for example. (See FIGS. 6-8.) Cs and G do not form a stable base-pairing interaction, and may, in fact, repel one another.

In some embodiments, blocking agents may contain nucleic acid analog segments comprising sets of purines and pyrimidines that do not form stable base pairs. The arrangement of such bases can serve to minimize interactions between different blocking agents in an assay. An example is a set of blocking agents made from stretches of Cs, G, D, and U2s. Cs and G, as well as U2s and D bases each bind to other nucleobases, for example, but repel each other. (See FIGS. 6-8.) Bases with one-less hydrogen bond upon pairing than their corresponding natural base-pairs may also be incorporated to weaken interactions between blocking agents; i.e. P and I.

These rules provide an information encoding system that is expanded in comparison to use of natural base in that bases pair with either one (Cs, Gs, U2s, U4s, D), two (A, IsoA, C, G) or three other bases (I, P, U) with the same (A:U2s=A:U) or different affinity (D:U>A:U). For example, the following tables show sets of bases that interact and repel, which can be used to construct a series of blocking agents and detection reagents such that a given set of partially complementary sample nucleic acids are blocked by the blocking agents.

| Base Pairs | Diaminopurine | Adenine |
|---|---|---|
| Uracil | 3 H-bonds | 2 H-bonds |
| 2-ThioUracil | repulsion | 2 H-bonds |
| Base Pairs | Diaminopurine | isoAdenine |
| Uracil | 3 H-bonds | 2 H-bonds |
| 4-ThioUracil | repulsion | 2 H-bonds |
| Base Pairs | Guanine | Inosine |
| Cytosine | 3 H-bonds | 2 H-bonds |
| 2-ThioCytosine | repulsion | 2 H-bonds |
| | | 2-oxo- |
| Base Pairs | Cytosine | Pyrimidine |
| Guanine | 3 H-bonds | 2 H-bonds |
| ThioGuanine | repulsion | 2 H-bonds |
| Base Pairs | isoGuanine | 2-oxo-Purine |
| isoCytosine | 3 H-bonds | 2 H-bonds |
| isoThioCytosine | repulsion | 2 H-bonds |

The non-natural bases disclosed herein can be made using synthesis techniques well known in the art, although some example syntheses are also provided herein.

### Nucleic Acid Backbones

The instant blocking agents may be made from DNA or RNA, or could be made from non-natural nucleic acid backbone units. Such non-natural backbone units include, but are not limited to, for example, PNA's, LNA's or phosphorothioate or 2′O-methyl nucleosides.

For example, in some embodiments, one or more phosphate oxygens may be replaced by another molecule, such as sulfur. In other embodiments, a different sugar or a sugar analog may be used, for example, one in which a sugar oxygen is replaced by hydrogen or an amine, or an O-methyl. In yet other embodiments, nucleic acid analog segments comprise synthetic molecules that can bind to a nucleic acid or nucleic acid analog. For example, a nucleic acid analog may be comprised of peptide nucleic acids (PNAs), locked nucleic acids (LNAs), or any derivatized form of a nucleic acid. Such backbone units may be attached to any base, including the natural bases A, C, G, T, and U, and non-natural bases.

As used herein, “peptide nucleic acid” or “PNA” means any oligomer or polymer comprising at least one or more PNA subunits (residues), including, but not limited to, any of the oligomer or polymer segments referred to or claimed as peptide nucleic acids in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470 6,201,103, 6,228,982 and 6,357,163; all of which are herein incorporated by reference.

The term PNA also applies to any oligomer or polymer segment comprising one or more subunits of the nucleic acid mimics described in the following publications: Lagriffoul et al., Bioorganic & Medicinal Chemistry Letters, 4: 1081-1082 (1994); Petersen et al., Bioorganic & Medicinal Chemistry Letters, 6: 793-796 (1996); Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et al., J. Chem. Soc. Perkin Trans. 11: 547-554 (1997); Lowe et al., J. Chem. Soc. Perkin Trans. 1 1:5 55-560 (1997); Howarth et al., J. Org. Chem. 62: 5441-5450 (1997); Altmann, K-H et al., Bioorganic & Medicinal Chemistry Letters, 7: 1119-1122 (1997); Diederichsen, U., Bioorganic & Med. Chem. Lett., 8: 165-168 (1998); Diederichsen et al., Angew. Chem. Int Ed., 37: 302-305 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMs) of Shah et al. as disclosed in WO96/04000.

As used herein, the term “locked nucleic acid” or “LNA” means an oligomer or polymer comprising at least one or more LNA subunits. As used herein, the term “LNA subunit” means a ribonucleotide containing a methylene bridge that connects the 2′-oxygen of the ribose with the 4′-carbon. See generally, Kurreck, Eur. J. Biochem., 270:1628-44 (2003).

Nucleic acid segments may be synthesized chemically or produced recombinantly in cells (see e.g. Sambrook et al. (1989) Molecular Cloning: A Laboratory Manual, 2nd ed. Cold Spring Harbor Press). Methods of making PNAs and LNAs are also known in the art (see e.g. Nielson, 2001, Current Opinion in Biotechnology 12:16; Sorenson et al. 2003, Chem. Commun. 7(17):2130).

The use of different nucleic acid backbones such as PNA or LNA further can affect the relative stability of different components of the system, and can also alter the charge of the different components, hence favoring or disfavoring certain hybridizations over others. For example, PNA-DNA and LNA-DNA duplexes are thermodynamically more stable than DNA-DNA duplexes, in general. Furthermore, one can also prevent two blocking agents from interacting with each other, while allowing them nonetheless to bind to complementary DNA or RNA sequences by modifying their charges. If the probes contain two positively charged nucleosides at a pairing location, those nucleosides will repel one another. But each will have high affinity for a DNA or RNA, which is negatively charged and presents a favorable charge-charge interaction.

### Kinetics and Thermodynamics

In some embodiments, the blocking agents may bind to nucleic acids in the sample with superior kinetics compared to the detection reagents. For instance, if the blocking agents are smaller than detection reagents, such as probes, the blocking agents may reach the nucleic acids in the sample faster than the detection reagents. Hence, kinetics may favor binding of the blocking agents over that of the detection reagents. The system may also be designed in some embodiments such that the probe, detection unit, or other detection reagents bind to nucleic acids in a sample with lower affinity than the blocking agents. Accordingly, binding of the blocking agents may also be thermodynamically favored. Thus, one of ordinary skill in the art may optimize a detection assay by controlling the reaction kinetics as well as temperature and buffer conditions.

In some embodiments, a detection reagent such as a probe may be designed to hybridize specifically to a particular target DNA or RNA sequence in a sample. Nevertheless, in some cases, that same sequence may appear in other non-target DNAs or RNAs in a sample. Blocking agents with favorable kinetics might bind to the non-target sequences faster than to the target sequences. In other cases, the probe may have weak affinity for partially complementary DNA or RNA sequences that might also be present in the sample. Blocking agents may also be designed to block those non-target sequences in the sample by binding to them with higher affinity than the probe would bind to them, for example.

For example, in some embodiments, a detection assay includes two reagents that are intended to specifically hybridize via nucleic acid base pairing. If the two hybridizing strands of nucleic acid on those detection reagents have a weak affinity for partially complementary, natural DNA or RNA sequences in a sample, their specific hybridization during the detection assay might be compromised. The result might be reduced signal to noise or false positive signals. Blocking agents may be designed that (1) bind with higher relative affinity or with better kinetics to those natural, complementary DNA or RNA sequences in the sample than the detection reagents, but, (2) do not bind either to each other or to any of the detection reagents to a significant extent. Accordingly, in some embodiments, blocking agents may serve to block binding between detection reagents and the sample so that the detection reagents may mutually hybridize. Such non-natural nucleic acid blocking agents may improve the signal to noise or reduce false positive signals in some detection assays.

In some embodiments, where blocking agents comprising such nucleic acid analog segments have low affinity for the detection reagents, the blocking agents and detection reagents (i.e. probes or other nucleic acid-containing reagents) can be mixed together either before their addition to the sample or upon addition to the sample. Accordingly, in some such embodiments, blocking agents do not need to be added in a separate step.

In some embodiments, the blocking agents of the invention may even be designed to act as competitors for the specific binding between probes and target DNA or RNA in a sample, or between interacting components of a detection system, rather than merely to block unwanted interactions. In such embodiments, the blocking agents may compete for target binding with the detection reagents. In some such embodiments, competition between blocking agents and detection reagents may serve to improve the overall signal to noise in the assay.

### Design of Nucleic Acid and Nucleic Acid Analog Segments

In some embodiments, the blocking agent and the nucleic acid analog segments L1 and/or L2 on the detection reagents may be, for example, 6, 8, 10, or 12 bases long. However, they can also be considerably longer, such as up to 20-25 bases or longer than 50 nucleobases. Not all of the bases in the segments necessarily need to be modified to weaken or prevent interactions between certain segments. (See FIG. 6, for example.) Thus, throughout the length of nucleic acid analog segments such as the exemplary L1, L2, B1, and B2 segments depicted in FIG. 6, a few natural base pairs may remain.

When designing the sequences of blocking agents and the corresponding detection reagents for an assay, it is also helpful to consider the design of the complete oligonucleotide or segment. One may wish to avoid sequences that might form hairpin or aggregate. Hence, in some embodiments, it may be helpful to design the blocking nucleic acid analog segments by following the above base-pairing schemes to avoid interaction between the blockers while maintaining interactions with sample nucleic acids, and by also following a few additional sequence constraints. For instance, one may design blocking agents such that, if more than one blocking agent is used:

the nucleic acid analog segment of one of the blocking agents contains a stretch of 3-5 purines in a row, for improved non-specific nucleic acid affinity; but such that the blocking agent with the purine stretch contains at least one Gs residue within the stretch so that guanosine-based quadroplex self structures are avoided;

sequences giving rise to known self-structures such as hairpin loops are avoided, for example GNRA tetraloop sequences in an RNA-based blocker, or adjacent bases that may form 3 or more base-pairs separated by a small number or intervening bases, such as 3-4 (e.g. a sequence such as AAAwxyzTTT, etc.);

long, repetitive base sequences are avoided; and

at least one set of potential base pairs between each of the two or more blockers would be repulsive if the blockers were to hybridize, such as D-U and Gs-C.

However, in some embodiments, the blockers may fail to satisfy one or more of the above constraints, yet still function efficiently in a detection assay.

To assist design of non-interacting blocking agents according to this invention following the above guidelines, one may use an algorithm, for example, for optimization of the sequences both to design appropriate base-pairing and non-base-pairing base sets on opposing strands, but also to follow the above constraints in designing each nucleic acid analog segment.

Furthermore, to design detection reagents that do interact in the assay but that may have reduced interactions with nucleic acids in the sample, a somewhat different set of guidelines may be followed. For instance, non-natural bases may be included in each the sequences which have reduced affinities for A, T, U, G, and C bases, but that have strong affinities for other non-natural bases. Examples include Gs:P, Cs:I, and U4s:isoA.

### Hybridization and Complementarity of Base Pairs

Two different base pairs or nucleic acid molecules may specifically hybridize. For instance, nucleic acid segments that form stable base pairing interactions throughout their length are 100% complementary. In some cases, however, specific hybridization of nucleic acid molecules may occur between molecules that are only partially complementary so long as the non-pairing bases do not significantly disturb the pairing bases in the molecules. But, at a certain point, as complementarity drops, the non-pairing bases disturb base-pairing between complementary bases in the molecules, or even cause steric clashes that lead to repulsion, and the two molecules do not specifically hybridize. Non-specific interactions may yet occur between some generally non-complementary molecules, however due to hydrophobic stacking of bases. The conditions used to induce hybridization may affect the stability of the interactions between two nucleic acid segments.

In some embodiments, the chosen hybridization conditions are “stringent conditions,” meaning herein conditions for hybridization and washes under which nucleotide sequences that are significantly complementary to each other remain bound to each other. For example, under those conditions, as little as 6 continuous complementary base will generally suffice for PNA to bind to complementary DNA, as will partially complementary sequences with two or more complementary stretches of 5 bases each.

Specified conditions of stringency are known in the art and can be found in Current Protocols in Molecular Biology, John Wiley & Sons, Inc. (Ausubel et al. 1995 eds.), sections 2, 4, and 6 (hereby incorporated by reference). Additionally, specified stringent conditions are described in Sambrook et al. (1989) Molecular Cloning: A Laboratory Manual, 2nd ed. Cold Spring Harbor Press, chapters 7, 9, and 11 (hereby incorporated by reference).

In other embodiments, the chosen hybridization conditions are “high stringency conditions.” An example of high stringency hybridization conditions is hybridization in 4× sodium chloride/sodium citrate (SSC) at 65-70° C. or hybridization in 4×SSC plus 50% formamide at 42-50° C., followed by one or more washes in 1×SSC, at 65-70° C. It will be understood that additional reagents may be added to hybridization and/or wash buffers, e.g., blocking agents (BSA or salmon sperm DNA), detergents (SDS), chelating agents (EDTA), Ficoll, PVP, etc.

In yet other embodiments, the chosen conditions are “moderately stringent conditions.” Moderate stringency, as used herein, includes conditions that can be readily determined by those having ordinary skill in the art based on, for example, the length of the molecular entity or a specific nucleic acid pr nucleic acid analog segment. Exemplified conditions are set forth by Sambrook et al. Molecular Cloning: A Laboratory Manual, 2d ed. Vol. 1, pp. 1.101-104, Cold Spring Harbor Laboratory Press (1989) (hereby incorporated by reference), and include use of a prewashing solution of 5×SSC, 0.5% SDS, 1.0 mM EDTA (pH 8.0), hybridization conditions of 50% formamide, 6×SSC at 42° C. (or other similar hybridization solution, such as Stark's solution, in 50% formamide at 42° C.), and washing conditions of 60° C., 0.5×SSC, 0.1% SDS.

In some embodiments, the chosen conditions are “low stringency” conditions. Low stringency conditions may include, as used herein, conditions that can be readily determined by those having ordinary skill in the art based on, for example, the length of the molecular entity. Low stringency may include, for example, pretreating the segment for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% W/N dextran sulfate, and 5-20×10⁶ CPM probe is used. Samples are incubated in hybridization mixture for 18-20 hours at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C.

Because the use of non-natural bases allows a wider range of base-pairing interactions, the binding affinities between detection reagents, blocking agents, and nucleic acids of a sample may be fine-tuned more precisely than through the use of only natural A, C, G, T, or U bases. Stronger pairs of non-natural nucleic acids can also produce faster detection protocols run at elevated temperatures such as 37° C. In extreme cases, such as where a probe comprises an antibody coupled to a non-natural nucleic acid sequence, one might utilize temperatures even up to and exceeding (under pressure) 100° C. At that temperature, the sample nucleic acids and the nucleic acids of the detection reagents may be completely denatured. In some embodiments, the blocking agents may block the nucleic acids in the sample even at such elevated temperatures or may block the sample nucleic acids first at lower temperatures such as 37° C., where the antibody probe and its target bind to each other.

### Sets of Nucleic Acid Analogs, Kits, and Detection Methods

Some embodiments of the invention include, for example, a set of nucleic acid segments comprising:

a first and a second nucleic acid segment, each optionally comprising at least one non-natural base, wherein the first and second segments specifically hybridize to each other, and wherein the first and second segments specifically hybridize to at least one target and/or non-target nucleic acid segment in a sample and

a third and a fourth nucleic acid segment, each comprising at least one non-natural base, wherein the third and fourth segments do not specifically hybridize to each other, and wherein the third and fourth segments specifically hybridize to at least one natural nucleic acid segment in the sample.

For example, in such a set, the first and second nucleic acid segments may act as detection reagents designed to specifically hybridize in a detection assay. For instance, those segments may comprise parts of detection reagents such as probes, amplification layers, and molecular entities carrying detectable labels. Those segments may also be nucleic acid analogs, comprising at least one non-natural base and/or at least one non-natural backbone unit such as PNA or LNA, for example.

The third and fourth nucleic acid analog segments may act as blocking agents which, for instance, do not significantly hybridize to each other, but which nonetheless do specifically hybridize to a set of natural nucleic acid sequences that could potentially be present in a sample. (See, for example, FIG. 6.) In some embodiments, the third and fourth segments may specifically hybridize to complementary sequences of DNA or RNA present in a sample, either natural or non-natural.

In some embodiments, none of the four segments hybridize to each other to a significant extent, while in others, there may be some interaction between the blocking agents (three and four) and the detection reagents (one and two). In some cases, the natural nucleic acid sequences to which the third and fourth segments hybridize could be similar to or the same as the sequences to which the first and second segments hybridize, thus setting up a competition for binding to those sequences among the nucleic acid analog segments of the set. Such competition could make a detection assay more stringent, for example.

One of more of the nucleic acid analog segments in such a set may comprise any of the non-natural bases described previously and may also include a non-natural backbone, such as at least one PNA or LNA backbone unit. The segments may also be entirely comprised of PNA or LNA, or other non-natural backbone (i.e. 2′O-methyl, phosphorothioate, etc.).

In other embodiments, the first and second and/or third and fourth nucleic acid segments may have a wide variety lengths, including as little as 6 and greater than 50 nucleotides. In some embodiments, any or all of the four nucleic acid segments may comprise non-natural bases such as D and Cs, which form repulsive pairs with U2s and G, but which form stable base pairs with U and I, respectively. The chart above and information in FIGS. 6-8 illustrate further sets of bases and base-pairs that may be used in nucleic acid segments of the invention.

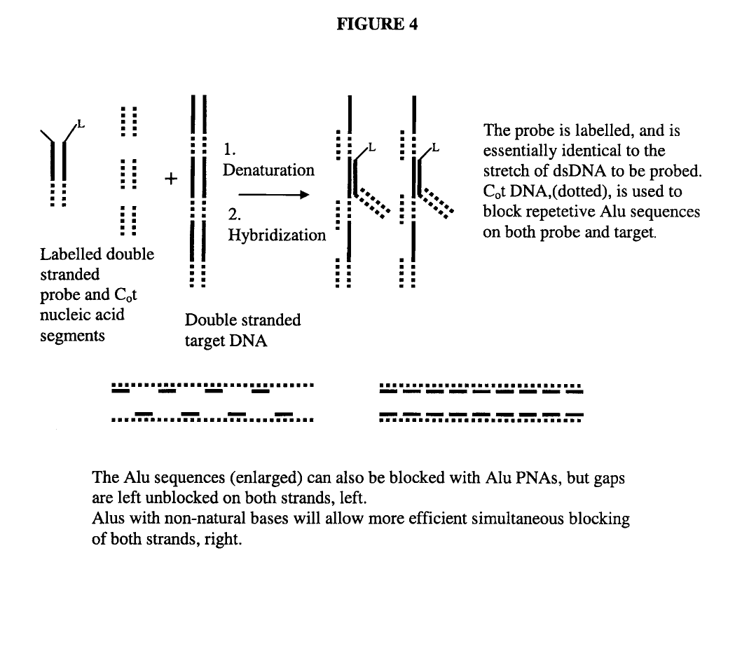

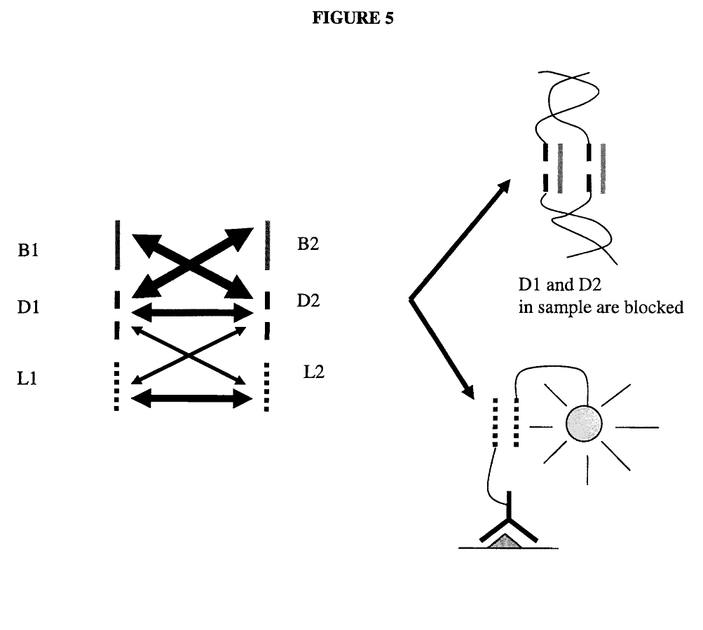

In some embodiments, the blocking agents (i.e. the third and fourth nucleic acid segments) may specifically hybridize to one or more genomic repeat sequences (i.e. see FIG. 4), such as Alu, Kpn, di-nucleotide, tri-nucleotide, penta-nucleotide, and hexa-nucleotide repeat sequences. In some embodiments, due to the expanded set of nucleobases they may comprise, the blocking agents may bind to those repetitive sequences while not have as repetitive a sequence as the nucleic acids they specifically hybridize to.

The instant invention also provides kits comprising the above sets of nucleic acid analog segments. Such kits could, for instance, form portions of biological or chemical detection kits. Thus, in addition to the sets of nucleic acid analog segments, the kits may contain other detection reagents, buffers, control samples, and instruction sheets or manuals, as needed in order to visualize the presence of one or more targets in a sample. Such kits could be used to carry out a variety of detection assays, such as in situ assays or blotting assays, for example.

Methods according to the invention include a method of detecting at least one target in a sample, comprising:

obtaining the sample potentially containing the target for detection,

incubating the sample with a first nucleic acid segment, or a molecular entity comprising said first nucleic acid segment,
wherein the first nucleic acid segment recognizes the target in the sample,

incubating the sample with a second nucleic acid segment which specifically hybridizes to the first nucleic acid segment,

incubating the sample with a third and a fourth nucleic acid segment, wherein the third and fourth nucleic acid segments are nucleic acid analog segments comprising at least one non-natural nucleobase, wherein the third and fourth segments do not specifically hybridize to each other, and wherein the third and fourth segments specifically hybridize to at least one set of complementary, natural nucleic acid segments;

adding further reagents serving to visualize the target in the sample; and

visualizing the target in the sample.

In some embodiments, the first and second nucleic acid segments may bind to complementary target sequences in a sample, such as the double-stranded DNA of genomic target sequences. In other embodiments, the first and second nucleic acid segments may be designed to specifically hybridize in the assay. For instance, they may serve to link a target to a detectable label, such as, by linking a molecular entity containing a probe to an amplification layer or a detectable label, or by linking an amplification layer to a detectable label. The first and second segments may also be nucleic acid analog segments, for example, with at least one non-natural nucleobase or with a non-natural nucleic acid backbone.

The third and fourth segments may be designed such that they specifically hybridize to complementary natural nucleic acid sequences that the first and second nucleic acid analog segments might otherwise non-specifically or specifically hybridize to in a sample. Hence, the third and fourth segments may serve to block such unwanted interactions in the assay. They may also be designed such that, despite interacting with complementary nucleic acid sequences potentially found in a sample, they do not specifically hybridize to each other.

In some embodiments, the third and fourth segments may block interactions between the first and second segments and the sample, but do not specifically hybridize to a significant extent with the first and second segments or with each other. Yet, in other embodiments, the third and fourth segments may hybridize specifically to the first and second segments. In such a case, the third and fourth segments could be added to the sample before the first and second segments.

In some embodiments, the third and fourth segments block natural nucleic acid sequences to which the first and/or second nucleic acid segments may specifically or non-specifically hybridize, such as sequences with full or partial complementarity to the first or second segments. In some embodiments, the third and fourth nucleic acid segments may serve to block genomic repeat sequences found in the sample, as well as potentially on the first and second nucleic acid segments, thus freeing the unique sequences of the first and second segments to specifically hybridize to their intended targets. In other embodiments, the third and fourth nucleic acid analog segments may compete for binding between the target(s) in the sample and the first and second nucleic acid analog segments, potentially enhancing the stringency of the detection assay.

The kits and methods according to the invention may be applied to any of a number of biological and chemical detection assays. Examples include in situ hybridization (ISH), immunohistochemistry (IHC), immunocytochemistry (ICC), flow cytometry, enzyme immuno-assays (EIA), enzyme linked immuno-assays (ELISA), Western, Southern, and Northern blots, as well as methods of labeling inside electrophoresis systems or on surfaces or arrays, and precipitation methods, among others.

In all of the embodiments above, the nucleic acid analog segments and natural nucleic acid segments may be isolated molecules or may form portions of larger molecular entities such as conjugates with polymers or proteins. Moreover, the embodiments above all allow for yet larger sets of nucleic acid analog segments containing additional blocking agents or detection reagents. For example, more than one blocking segment could be used with each detection reagent segment, or vice versa. Further, in some experiments, more than one target may be the subject of detection.

The following non-limiting examples serve to illustrate a few, particular aspects of the instant invention.

### WORKING EXAMPLES

### Example 1

### Preparation of Pyrimidinone-Monomer

1. In dry equipment 4.6 g of solid Na in small pieces was added to 400 mL ethanol (99.9%), and was dissolved by stirring. Hydroxypyrimidine hydrochloride, 13.2 g, was added and the mixture refluxed for 10 minutes. Then 12.2 mL ethyl-bromoacetate (98%) was added and the mixture refluxed for 1½ hour. The reaction was followed using Thin Layer Chromatography (TLC). The ethanol was evaporated leaving a white compound, which was dissolved in a mixture of 80 mL of 1M NaCltrate (pH 4.5) and 40 mL of 2M NaOH. This solution was extracted four times with 100 mL Dichloromethane (DCM). The DCM phases were pooled and washed with 10 mL NaCltrate/NaOH-mixture. The washed DCM phases were evaporated under reduced pressure and resulted in 17.2 g of crude solid product. This crude solid product was recrystallized with ethylacetate giving a yellow powder. The yield for this step was 11.45 g (63%).

2. The yellow powder, 12.45 g. from above was hydrolyzed by refluxing overnight in a mixture of 36 mL DIPEA, 72 mL water and 72 mL dioxane. The solvent was evaporated and water was removed from the residue by evaporation from toluene. The yield for this step was 100%.

3. OBS. Pyrimidinone acetic acid (10.5 g), 16.8 g PNA-backbone ethylester, 12.3 g DHBT-OH, 19 mL Triethylamine was dissolved in 50 mL N,N-dimethylformamide (DMF). DIPIDIC (11.8 mL) was added and the mixture stirred overnight at room temperature. The product was taken up in 100 mL DCM and extracted three times with 100 mL of dilute aqueous NaHCO₃. The organic phase was extracted twice with a mixture of 80 mL of 1M NaCltrate and 20 mL of 4M HCl. Because TLC showed that some material was in the citrate phase, it was extracted twice with DCM. The organic phases were pooled and evaporated. Because there was a precipitation of urea, the product was dissolved in a DCM, and the urea filtered off. Subsequent evaporation left an orange oil. Purification of the orange oil was performed on a silica column with 10% methanol in DCM. The fractions were collected and evaporated giving a yellow foam. The yield for this step was 7.0 g (26.8%).

4. The yellow foam (8.0 g) was hydrolyzed by reflux overnight in 11 mL DIPEA, 22 mL water, and 22 mL dioxane. The solvent was evaporated and the oil was dehydrated by evaporation from toluene leaving an orange foam. The yield for this step was 100%.

### Example 2

### Preparation of the Thio-Guanine Monomer

1. 6-Chloroguanine (4.93 g) and 10.05 g K₂CO₃ was stirred with 40 mL DMF for 10 minutes at room temperature. The reaction mixture was placed in a water bath at room temperature and 3.55 mL ethyl bromoacetate was added. The mixture was stirred in a water bath until TLC (20% Methanol/DCM) showed that the reaction was finished. The precipitated carbonate was filtered off and washed twice with 10 mL DMF. The solution, which was a little cloudy, was added to 300 ml water, whereby it became clear. On an ice bath the target compound slowly precipitated. After filtration the crystals were washed with cold ethanol and dried in a desiccator. The yield for this step was 3.3 g (44.3%) of ethyl chloroguanine acetate.

2. Ethyl chloroguanine acetate (3.3 g) was dissolved by reflux in 50 mL absolute ethanol. Thiourea (1.08 g) was added. After a refluxing for a short time, precipitate slowly began forming. According to TLC (20% Methanol/DCM) the reaction was finished in 45 minutes. Upon completion, the mixture was cooled on an ice bath. The precipitate was then filtered and dried overnight in a desiccator. The yield for this step was 2.0 g (60%) ethyl thioguanine acetate.

3. Ethyl thioguanine acetate (3.57 g) was dissolved in 42 mL DMF. Benzylbromide (2.46 mL) was then added and the mixture stirred in an oil bath at 45° C. The reaction was followed using TLC (25% Methanol/DCM). After 3 hours all basis material was consumed. The step 3 target compound precipitated upon evaporation under reduced pressure and high temperature. The precipitate was recrystallized in absolute ethanol, filtered and then dried in a desiccator. The yield for this step was 3.88 g (82%) of methyl benzyl thioguanine ethylester.

4. Methyl benzyl thioguanine ethylester (5.68 g) was dissolved in 12.4 mL of 2M NaOH and 40 mL THF, and then stirred for 10 minutes. The THF was evaporated by. This was repeated. The material was dissolved in water and then 6.2 mL of 4M HCl was added, whereby the target product precipitated. Filtering and drying in a desiccator. The yield for this step was 4.02 g (77%).

5. The product of step 4 (4.02 g), 3.45 g backboneethylester, 9 mL DMF, 3 mL pyridine, 2.1 mL triethylamine and 7.28 g PyBop were mixed and then stirred at room temperature. After 90 minutes a solid precipitation formed. The product was taken up in 125 mL DCM and 25 mL methanol. This solution was then extracted, first with a mixture of 80 mL of 1M NaCltrate and 20 mL of 4M HCl, and then with 100 mL dilute aqueous NaHCO₃. Evaporation of the organic phase gave a solid material. The material was dissolved in 175 mL boiling ethanol. The volume of the solution was reduced to about 100 mL by boiling. Upon cooling in an ice bath, the target product precipitate. The crystals were filtered, washed with cold ethanol and then dried in a desiccator. The yield of this step was 6.0 g (86%.)

6. The product of step 5 (6.0 g) was dissolved in 80 mL THF, 7.5 mL 2M NaOH and 25 mL water. The solution became clear after ten minutes of stirring. THF was evaporated. Water (50 mL) was added to the mixture. THF was evaporated. Water (50 mL) was added to the mixture. When the pH was adjusted by the addition of 3.75 mL of 4M HCl, thio-guanine monomer precipitated. It was then filtered, washed with water and dried in a desiccator. The yield for this step was 5.15 g (91%).

### Example 3

### Preparation of Diaminopurine Acetic Acid Ethyl Ester

1. Diaminopurine (10 g) and 40 g of K₂CO₃ were added to 85 mL of DMF and stirred for 30 minutes. The mixture was cooled in a water bath to 15° C. Ethyl bromoacetate (3 mL) was added three times with 20 minute intervals between each addition. This mixture was then stirred for 20 minutes at 15° C. The mixture was left in the water bath for another 75 minutes, and the temperature increased to 18° C. The DMF was removed by filtering and the remaining K₂CO₃ was added to 100 mL of ethanol and refluxed for 5 minutes. Filtering and repeated reflux of the K₂CO₃ in 50 mL ethanol, filtering. The pooled ethanol phases were placed in a freezer, after which crystals formed. These crystals were filtered, washed with cold ethanol, filtered again and then dried in a desiccator overnight. The yield for this step was 12 g (76%).

### Example 4

### Suppression of Non-Specific Background Staining by Addition of a PNA Blocker

Detection reagents comprising nucleic acid analog segments are applied on FFPE tissue without a primary reagent. The resulting DAB stain is therefore all non-specific.

A nucleic acid analog segment with a PNA backbone was coupled to dextran and HRP (195-157) was diluted to final concentration of 0.05 μM (based on detection of dextran) in BAP-HEPES-buffer (1.5% BSA, 3% PEG, 0.15M NaCl, 0.05% 4-aminoantipyrine, 10 mM HEPES, pH 7.2) and was applied on a multi-tissue section. Following 10 minutes incubation at room temperature (RT) the section was washed 5 minutes using 10×-diluted S3006 buffer (available from Dako Denmark A/S).

DAB+ working solution (Dako K3468) was then applied. Following 10 minutes incubation, the sections were washed 5 minutes deionized water. Finally the sections were counter-stained 5 minutes using haematoxylin S3301 (Dako), rinsed in deionized water, washed 3 minutes in a wash buffer, and mounted in Faramount S3025 (Dako).

The conjugate (195-157) resulted in non-specific background staining at a level of 3+.

A nucleic acid analog segment blocker (195-161) was diluted to a final concentration of 2 μM in BP-HEPES-buffer (1.5% BSA, 3% PEG, 0.15M NaCl, 10 mM HEPES, pH 7.2) and was then applied to the sample. Following 10 minutes incubation at RT the section was washed 5 minutes using 10× diluted S3006 buffer (Dako).

The detection reagent described above (195-157) was diluted to final concentration of 0.05 μM (dextran) in BAP-HEPES-buffer and was applied to the sample. Following 10 minutes incubation at RT, the section was washed 5 minutes using 10× diluted S3006 (Dako). DAB+ working solution (Dako K3468) was applied. Following 10 minutes incubation, the sections were washed 5 minutes deionized water. Finally the sections were counter-stained 5 minutes using haematoxylin S3301 (Dako), rinsed in deionized water, washed 3 minutes in a wash buffer, and mounted in Faramount S3025 (Dako).

The addition of the blocker (195-161) reduces the non-specific background staining from 3+ to 2+.

### ADDITIONAL EXAMPLES

Further non-limiting example embodiments include the following:

### Example 5

Set of nucleic acid segments comprising:

a first and a second nucleic acid segment, wherein the first and second segments specifically hybridize to each other, and wherein the first and second segments specifically hybridize to at least one target and/or at least one non-target natural nucleic acid segment present in a sample; and

a third and a fourth nucleic acid segment, each comprising at least one non-natural base, wherein the third and fourth segments do not specifically hybridize to each other, and wherein the third and fourth segments specifically hybridize to at least one natural nucleic acid segment present in the sample.

The set of Example 5, wherein at least one of the first and second nucleic acid segments comprise more than 50 nucleobases.

The set of Example 5, wherein at least one of the third and fourth nucleic acid segments comprise at least five consecutive nucleobases complementary to at least one of the natural nucleic acid segments in the sample.

The set of Example 5, wherein at least one of the third and fourth nucleic acid segments comprise from about 5 to about 50 nucleobases. The set above, wherein at least one of the third and fourth nucleic acid segments comprise from about 12 to about 25 nucleobases.

The set of Example 5, wherein at least one of the first and second nucleic acid segments comprise DNA or non-natural backbone units.

The set of Example 5, wherein the third and/or fourth nucleic acid segments comprise at least one peptide-nucleic acid (PNA) backbone unit.

The set of Example 5, wherein the at least one non-natural base is diaminopurine (D) or thiocytosine (Cs). The set above, wherein the third and fourth nucleic acid analog segments together comprise at least one D and U or at least one Cs and G base-pairing.

The set of Example 5, wherein at least one of the third and fourth nucleic acid segments specifically hybridize to non-target nucleic acid segments in the sample.

The set of Example 5, wherein at least one of the first nucleic acid segments and at least one of the third nucleic acid segments hybridize to each other.

The set of Example 5, wherein at least one of the second nucleic acid segments and at least one of fourth nucleic acid segments hybridize to each other.

The set of Example 5, wherein at least one of the first and second nucleic acid segments further comprise at least one detectable label.

The set of Example 5, wherein the third and fourth segments specifically hybridize to complementary strands of natural nucleic acid segments in the sample.

The set of Example 5, wherein the target natural nucleic acid segment is a genetic locus optionally comprising at least one genomic repeat sequence.

The set of Example 5, wherein the non-target natural nucleic acid segments in the sample comprise genomic repeat sequences.

The set of Example 5, wherein the first and/or second nucleic acid segments comprise genomic repeat sequences. The set above, wherein the repeat sequences comprise one or more of Alu repeats, Kpn repeats, di-nucleotide repeats, tri-nucleotide repeats, penta-nucleotide repeats, and hexa-nucleotide repeats.

The set of Example 5, wherein the third and fourth segments specifically hybridize to genomic repeat sequences in the sample, and optionally specifically hybridize to genomic repeat sequences in the first and second segments.

A kit comprising the first, second, third, and fourth nucleic acid segments of Example 5, or any of the modifications of Example 5 described above. The kit above, further comprising reagents for visualization of at least one target in a sample. A method of detecting a target in a sample, comprising using such a kit.

### Example 6

A set of nucleic acid analog segments comprising:

a first and a second nucleic acid analog segment, each comprising at least one non-natural base, wherein the first and second segments specifically hybridize to each other; and

a third and a fourth nucleic acid analog segment, each comprising at least one non-natural base, wherein the third and fourth segments do not specifically hybridize to each other, and wherein the third and fourth segments specifically hybridize to at least one set of complementary, natural nucleic acid segments in a sample.

The set of Example 6, wherein at least one of the first, second, third and fourth nucleic acid analog segments comprises at least five consecutive nucleobases which are complementary to at least one of the natural nucleic acid segments present in the sample.

The set of Example 6, wherein at least one of the first and second nucleic acid analog segments specifically hybridizes to at least one of the natural nucleic acid segments.

The set of Example 6, wherein the first and/or second nucleic acid analog segments comprise at least one peptide-nucleic acid (PNA) backbone unit.

The set of Example 6, wherein the third and/or fourth nucleic acid analog segments comprise at least one PNA backbone unit.

The set of Example 6, wherein the at least one non-natural base is diaminopurine (D) or thiocytosine (Cs). The set above, wherein the third and fourth nucleic acid analog segments together comprise at least one D and U or at least one Cs and G base-pairing.

A kit comprising the first, second, third, and fourth nucleic acid analog segments of Example 6. The kit above, further comprising reagents for visualization of at least one target in a sample. A method of detecting a target in a sample, comprising using the kit above.

What is claimed is:

1. A set of nucleic acid segments comprising:
a first and a second nucleic acid segment, wherein the first and second segments specifically hybridize to each other, and wherein the first and second segments specifically hybridize to at least one target and/or at least one non-target natural nucleic acid segment present in a sample; and
a third and a fourth nucleic acid segment, each comprising at least one non-natural base, wherein the third and fourth segments do not specifically hybridize to each other, and wherein the third and fourth segments specifically hybridize to at least one natural nucleic acid segment present in the sample.

2. The set of claim 1, wherein at least one of the first and second nucleic acid segments comprise more than 50 nucleobases.

3. The set of claim 1, wherein at least one of the third and fourth nucleic acid segments comprise at least five consecutive nucleobases complementary to at least one of the natural nucleic acid segments in the sample.

4. The set of claim 1, wherein at least one of the third and fourth nucleic acid segments comprise from about 5 to about 50 nucleobases.

5. The set of claim 4, wherein at least one of the third and fourth nucleic acid segments comprise from about 12 to about 25 nucleobases.

6. The set of claim 1, wherein at least one of the first and second nucleic acid segments comprise DNA or non-natural backbone units.

7. The set of claim 1, wherein the at least one non-natural base is diaminopurine (D) or thiocytosine (Cs).

8. The set of claim 7, wherein the third and fourth nucleic acid analog segments together comprise at least one D and U and/or at least one Cs and G base-pairing.

9. The set of claim 1, wherein the third and/or fourth nucleic acid segments comprise at least one peptide-nucleic acid (PNA) backbone unit.

10. The set of claim 1, wherein at least one of the third and fourth nucleic acid segments specifically hybridize to non-target nucleic acid segments in the sample.

11. The set of claim 1, wherein at least one of the first nucleic acid segments and at least one of the third nucleic acid segments hybridize to each other.

12. The set of claim 1, wherein at least one of the second nucleic acid segments and at least one of fourth nucleic acid segments hybridize to each other.

13. The set of claim 1, wherein at least one of the first and second nucleic acid segments further comprise at least one detectable label.

14. The set of claim 1, wherein the third and fourth segments specifically hybridize to complementary strands of natural nucleic acid segments in the sample.

15. The set of claim 1, wherein the target natural nucleic acid segment is a genetic locus optionally comprising at least one genomic repeat sequence.

16. The set of claim 1, wherein the non-target natural nucleic acid segments in the sample comprise genomic repeat sequences.

17. The set of claim 1, wherein the first and/or second nucleic acid segments comprise genomic repeat sequences.

18. The set of claim 17, wherein the genomic repeat sequences comprise one or more of Alu repeats, Kpn repeats, di-nucleotide repeats, tri-nucleotide repeats, penta-nucleotide repeats, or hexa-nucleotide repeats.

19. The set of claim 1, wherein the third and fourth segments specifically hybridize to genomic repeat sequences in the sample, and optionally specifically hybridize to genomic repeat sequences in the first and second segments.

20. A kit comprising the first, second, third, and fourth nucleic acid segments of claim 1.

21. The kit of claim 20, further comprising reagents for visualization of at least one target in a sample.

22. Use of the set of claim 1 to detect at least one target in a sample.

23. A method of detecting at least one target in a sample comprising incubating the sample with the set of claim 1.

24. A set of nucleic acid analog segments comprising:
a first and a second nucleic acid analog segment, each comprising at least one non-natural base, wherein the first and second segments specifically hybridize to each other; and
a third and a fourth nucleic acid analog segment, each comprising at least one non-natural base, wherein the third and fourth segments do not specifically hybridize to each other, and wherein the third and fourth segments specifically hybridize to at least one set of complementary, natural nucleic acid segments in a sample.

25. The set of claim 24, wherein at least one of the first, second, third, and fourth nucleic acid analog segments comprises at least five consecutive nucleobases that are complementary to at least one of the natural nucleic acid segments present in the sample.

26. The set of claim 24, wherein at least one of the first and second nucleic acid analog segments specifically hybridizes to at least one of the natural nucleic acid segments.

27. The set of claim 24, wherein the first and/or second nucleic acid analog segments comprise at least one peptide-nucleic acid (PNA) backbone unit.

28. The set of claim 24, wherein the third and/or fourth nucleic acid analog segments comprise at least one PNA backbone unit.

29. The set of claim 24, wherein the at least one non-natural base is diaminopurine (D) or thiocytosine (Cs).

30. The set of claim 29, wherein the third and fourth nucleic acid analog segments together comprise at least one D and U and/or at least one Cs and G base-pairing.

31. A kit comprising the first, second, third, and fourth nucleic acid analog segments of claim 24.

32. The kit of claim 31, further comprising reagents for visualization of at least one target in a sample.

33. Use of the kit of claim 31 to detect at least one target in a sample.

34. A method of detecting at least one target in a sample, comprising incubating the sample with the set of claim 24.

35. A set of nucleic acid analog segments comprising:
a first and a second nucleic acid analog segment, each comprising at least one non-natural base, wherein the first and second segments specifically hybridize;
a third and a fourth nucleic acid analog segment, each comprising at least one non-natural base and a peptide-nucleic acid (PNA) backbone,
wherein the third and fourth segments do not specifically hybridize to each other, and wherein the third and fourth segments specifically hybridize to at least one set of complementary, natural nucleic acid segments in a sample; and
wherein the third and fourth segments comprise at least one set of D and U and/or at least one set of Cs and G base pairings.
